(19) United States
(12) Plant Patent Application Publication
(10) Pub. No.: US 2021/0321550 P1
(43) Pub. Date: Oct. 14, 2021

(54) Mentha Plant Named 'Columbia'

(50) ;

(71) Applicant: WILD Flavors, Inc., Erlanger, KY (US)

(72) Inventors: Deven Holgram, Eugene, OR (US); Jie Liu, Corvallis, OR (US); Tim Chambers, Kalamazoo, MI (US); Tim Nemeth, Kalamazoo, MI (US); Mark Morris, Eugene, OR (US); A.J. Todd IV, Kalamazoo, MI (US); Rich Schneider, Sunnyside, WA (US); Mark Nelson, Caldwell, ID (US); Bryon Quebbeman, La Grande, OR (US)

(21) Appl. No.: 16/846,122

(22) Filed: Apr. 10, 2020

(51) Int. Cl. A01H 6/50

(52) U.S. Cl. PLT/259

(57) ABSTRACT

A new and distinct variety of Mentha x piperita, ‘Columbia’ is characterized by its unique oil chemistry and sweet complex flavor profile. The new varietal offers a Mentha x piperita variety that it is resistant to diseases such as Verticillium Wilt and Mint Rust.

Latin name of the genus and species of the plant claimed: Mentha x piperita.

Variety determination: ‘Columbia’.

### FIELD OF THE INVENTION

The present invention relates to a new and distinct peppermint plant botanically known as Mentha x piperita. The new variety has been named ‘Columbia’ and will be referred to as such herein.

### BACKGROUND OF THE INVENTION

Many commercial peppermint plants are susceptible to Verticillium wilt which can result in the loss of entire fields of peppermint plants. Since the vast majority of peppermint plants grow out of the rootstock of other peppermint plants, finding new peppermint plants that survive the Verticillium wilt disease is a challenge. While a few peppermint plants have been found that exhibit some Verticillium resistance, such plants do not produce commercially acceptable oils. What is needed is a peppermint plant that is resistant to Verticillium wilt, yet produces commercially acceptable oils.

### SUMMARY OF THE INVENTION

In each of its various embodiments, the present invention fulfills these needs and discloses a new peppermint plant that is resistant to Verticillium wilt and produces commercially acceptable oil.

The Mentha x piperita variety described herein is characterized by its unique oil chemistry and sweet complex flavor profile. This new variety has been named Columbia and will be referred to as such herein. It is a reduced cost, sustainable North American peppermint variety of which its essential oil is to be used in flavorings.

Columbia is a distinct Mentha x piperita variety that it is resistant to diseases such as Verticillium dahlia (Verticillium Wilt) and Puccinia menthae (Mint Rust) and has improved agronomic traits such as yield and stand longevity.

Columbia may be used in flavoring confections, pharmaceuticals, beverages, tobacco, and oral care products including but not limited to chewing gum, hard boiled candy, soft chews, pressed tablets, mint flavored chocolate, bakery, toothpaste, mouthwash, cough and throat lozenges, cigarettes and smokeless tobacco, and flavored beverages and syrups. Columbia is disease resistant, has a greater stand longevity and growth vigor, requires less crop rotation, and provides greater yields while enhancing the complexity of peppermint flavor profiles. These qualities give mint growers a more sustainable and profitable option over the typical peppermint varietal types currently used.

Columbia was derived from non-GMO techniques and is proven to be resistant to diseases such as Verticillium dahlia and Puccinia menthae, giving mint growers an advantage to reclaim once fertile land infected with Verticillium dahlia and Puccinia menthae. Other attempts have been made to produce peppermint plants that are resistant to these diseases, but Columbia has benefits that are highly desirable to both agricultural and flavor industries.

Through an advanced selective breeding program using non-GMO techniques, certain varieties of peppermint were selected for the plant breeding program based on specific traits. The propagated plant varieties were entered into growth trials exposed to disease and pests while being monitored for plant health and yield. Results of the trial work demonstrated and proved that key peppermint qualities discussed herein were present and repeatable throughout the growth cycles of these propagated varieties over multiple years through field plot trials. Field trial and greenhouse trials included exposing peppermint varietal plants to known disease in infected or inoculated soils. Field observations were recorded along with yield data at harvest. Analytical and sensory qualities were monitored and recorded.

Analytical results show an increase in certain flavor molecules, like esters and ketones, that improve the complexity of the flavor and organoleptic profiles of the oil from Columbia. The peppermint essential oil from Columbia is able to compliment or replace existing commercial varieties of Mentha piperita, like Black Mitcham. When the essential oil from Columbia is blended with a Black Mitcham peppermint oil, the blend exhibited a nice rounded, well-aged profile along with a slightly brighter, sweeter, more complex peppermint character.

The oil chemistry and flavor profile of ‘Columbia’ is sustained throughout its maturity when grown in the U.S. mint producing regions of Oregon, Washington, Idaho and the Midwest.

### BRIEF DESCRIPTION OF THE DRAWINGS

The accompanying color photographs of ‘Columbia’ shows the new variety.

FIG. 1 shows the flower spike of ‘Columbia.’

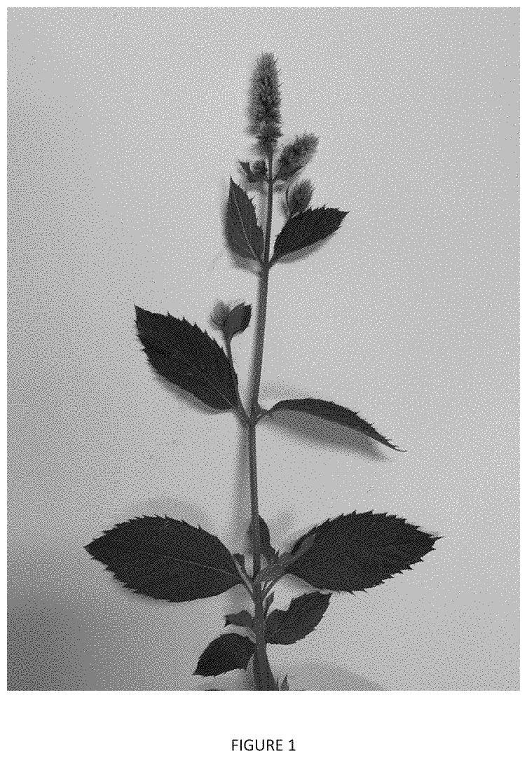

FIG. 2 shows the mature leaves of ‘Columbia.’

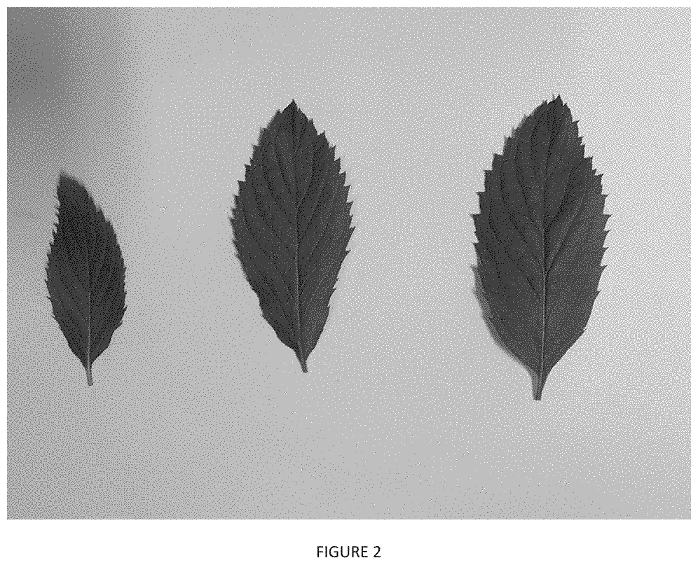

FIG. 3 shows the SNPs of Columbia compared other mint varieties.

### DETAILED DESCRIPTION OF THE INVENTION

Columbia is a unique and distinct proprietary North American peppermint variety. Columbia's essential oil finds utility in mint flavorings. Columbia is more sustainable and proven to be resistant to Verticillium Wilt and Mint Rust that is unlike any of the other commercial varieties of peppermint used in the industry today. Columbia is also an exceptionally vigorous plant and produces high quality peppermint oil, both in terms of chemical composition and organoleptic properties. Columbia is intended for commercial use as an alternative to disease susceptible varieties like Black Mitcham.

The preferred usage level of the essential oil from Columbia as a flavoring agent for use in flavoring confection, pharmaceutical, beverage, tobacco, and oral care products varies from 0.01 to 5.0% by weight, either by itself or in combination with other mint oils, components thereof, or natural and/or synthetic flavoring materials, in the finished product depending on the finished application. Usage levels may even be higher depending on the potential use based on the individual components of other flavor components such as 1-limonene, Menthyl acetate, Menthofuran, or others.

Oil Composition. Columbia contains unique chemical properties that can match and/or enhance the characteristics of Black Mitcham peppermint oil resulting in a complex profile to a flavor oil that is produced in a more sustainable manner while providing a better yielding alternative to the industry standard. The chemical properties of Columbia oil obtained from a gas ghromatography instrument equipped with a Flame Ionization Detector (GC-FID) include a 1,8-cineole content of 2.5 to 5.5%, 1-limonene from 1.0 to 3.0%, menthone from 20 to 45%, menthofuran from 0.2 to 3.5%, menthol 25 to 45%, menthyl acetate from 3.0 to 15%, and isopulegol of below 0.2%. These values are listed in area percentage.

The essential oil chemistry profile of ‘Columbia’ was compared to commercially available varieties of the M. piperita varities ‘Black Mitcham’ and Mentha canadensis (whole arvensis) and are shown in Table 1.

TABLE 1
M. x piperita ‘Columbia’ Comparison to Commercial
Varieties of Mint by Gas Chromatography

| Flavor | M. x piperita | M. piperita | M. canadensis |
|---|---|---|---|
| Compound | ‘Columbia’ | ‘Black Mitcham’ | Whole Arvensis |
| beta-Myrcene | 0.30 to 0.50% | 0.20 to 0.35% | 0.50 to 0.80% |
| 1,8-Cineole | 2.5 to 5.5% | 3.0 to 5.0% | <0.5% |
| trans-Sabinene | 0.2 to 1.0% | 0.8 to 2.0% | <0.1% |
| Hydrate | | | |
| l-Menthone | 20 to 45% | 15 to 22% | 6 to 12% |
| Menthofuran | 0.2 to 3.5% | 0.5 to 5.0% | <0.1% |
| d-Isomenthone | 1.5 to 3% | 2 to 4% | 3 to 5% |
| Menthyl Acetate | 3 to 15% | 3 to 6% | 1 to 4% |
| Isopulegol | <0.2% | <0.2% | 0.7 to 0.9% |
| Menthol | 25 to 45% | 33 to 49% | 62 to 72% |
| Pulegone | 0.2 to 3.0% | 0.5 to 3.0% | 0.5 to 1.0% |
| Germacrene D | 0.5 to 3.0% | 2.0 to 4.0% | 0.2 to 1.0% |
| Viridiflorol | <0.6% | 0.4 to 0.8% | <0.1 |

(All values listed are in area percentage)

Analytical results show an increase in certain molecules, like esters and ketones, that improves the complexity of the flavor and organoleptic profiles of Columbia that can compliment or replace existing commercial varieties of Mentha piperita such as like Black Mitcham. When the essential oil from Columbia is blended with a Black Mitcham peppermint oil, the blend exhibited a nice rounded, well-aged profile along with adding a slightly brighter, sweeter, more complex peppermint character.

The relative amounts of the flavor compounds of Table 1 are distinct and uniquely distinguishable from Black Mitchum and other known commercial peppermint cultivars. The combination of high menthone (>22%) and menthyl acetate (>5%) levels, the low ratio of menthol to menthyl acetate (<5:1), as well as the high ratio of 1-menthone to d-isomenthone (>12:1) are unique identifiers for the oil from the Columbia variety.

### BOTANICAL DESCRIPTION

‘Columbia’ is an herbaceous perennial with an upright shrubby growth habit. The plant spreads by stolons. It is reasonable for a single rooted cutting to spread and cover 0.2-0.4 square meters after one growing season. Numerous branching stems are produced each year with an eventual height up to 1 m at flowering.

Stem and Leaves.

Columbia has a square stem, 5-7 mm on a side near the base. Mature leaves are ovate to lanceolate, oppositely arranged on the stem with an obtuse base, an acute apex, serrate margin, and generally decreasing in size towards the apex of a blooming stem. Leaf surface color is medium green, with the ventral leaf surface, petiole, and stem being a slightly lighter green color. The base of the stem has a more “woody” appearance and quality with more brownish coloring. The fragrance is sweet peppermint-like.

Flower. Columbia possesses a terminal flower spike with only a few very small bract-like leaves only at the base of the inflorescence. This differs from M. canadensis varieties. The individual flowers contain a 4-lobed, nearly regular pale lilac corolla with a short tube, 8 mm long from the base of the calyx to the tip of the forked white stigma. The calyx generally has an average of five teeth fused at the base forming a short tube and is 3 mm long. The calyx, peduncle and pedicel colors are light green. The formation of seed is a rare event. There is no major flower fragrance.

Columbia has been grown in different field locations and under greenhouse conditions. The basic morphological characteristics have remained consistent, with minor differences easily attributed to differences in maturity, climate, soils, fertilizer, water regime, etc. Columbia is distinct from other mints in its characteristics.

A single-nucleotide polymorphism (SNP) is a substitution of a nucleotide that occurs at a specific position in the genome. SNPs are the most common form of genome variation. SNPs are abundant and widely distributed within the genome. Genotyping by sequencing (GBS) is a method to discover SNP in order to perform genotyping studies. Mint genomic DNA was extracted and digested using restriction enzyme ApeKI. Next generation sequencing is performed resulting in about 100 bp single end reads. Raw sequence data are filtered and are aligned to Mentha longifolia genome as a reference. A total of 344,711 quality SNPs was found and selected from 11 M. x piperita varieties by using GBS.

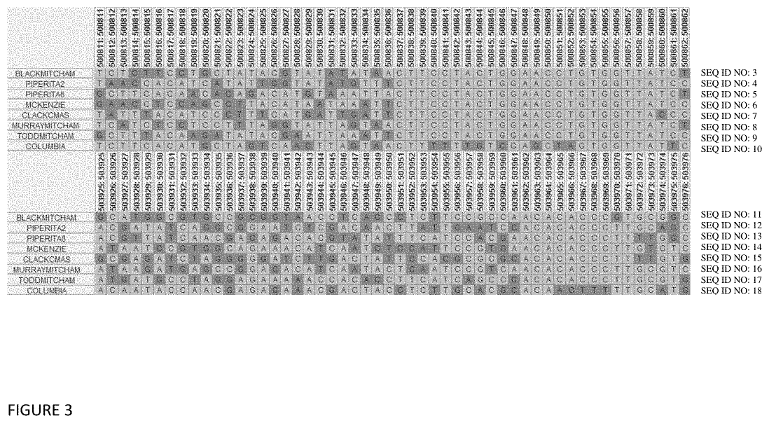

Examples of SNPs among 8 peppermint varieties are shown in FIG. 3. It revealed 14 SNPs from physical position 500840-500852 which reads TTTTGTGGAGCTA for Columbia vs CCTACTGGAACCT for all other peppermint varieties and from 503965-503969 which reads ACTTT for Columbia vs CACCC for all other peppermint varieties.

The above genetic description based on SNP markers indicated a close genetic relationship between Columbia with other peppermint varieties and unique genetic profiles of Columbia from other peppermint varieties and the SNP variations are within the different varieties of the same peppermint species M. x. piperita.

What is claimed is:

1. A new and distinct variety of Mentha x piperita named ‘Columbia’ substantially as shown and described.


SEQUENCE LISTING (18 sequences)

SEQ ID NO: 1 - DNA, 13 bp, Mentha x piperita
ttttgtggagcta

SEQ ID NO: 2 - DNA, 13 bp, Mentha x piperita
cctactggaacct

SEQ ID NO: 3 - DNA, 51 bp, Mentha x piperita
tctcttcctgctatacgtatatataacttcctactggaacctgtggttatc

SEQ ID NO: 4 - DNA, 51 bp, Mentha x piperita
taaccacatcatattggtatatgtttcttcctactggaacctgtggttatc

SEQ ID NO: 5 - DNA, 51 bp, Mentha x piperita
gcttcacaacacagacatgtaaattacttcctactggaacctgtggttatc

SEQ ID NO: 6 - DNA, 51 bp, Mentha x piperita
gaacctccagccttacataataaattcttcctactggaacctgtggttatc

SEQ ID NO: 7 - DNA, 51 bp, Mentha x piperita
tatttacatccctttcatgattgattcttcctactggaacctgtggttacc

SEQ ID NO: 8 - DNA, 51 bp, Mentha x piperita
tcatctcctcctttaggtattagtaacttcctactggaacctgtggttatc

SEQ ID NO: 9 - DNA, 51 bp, Mentha x piperita
gctttacaagatatacgaattaaattcttcctactggaacctgtggttatc

SEQ ID NO: 10 - DNA, 51 bp, Mentha x piperita
tcttcacatgctagtcaagttagtaacttttttgtcgagctagtggttatt

SEQ ID NO: 11 - DNA, 52 bp, Mentha x piperita
gcatggcgtgccgcggtaacctcaccctcttccgccaacacacccgtgcggc

SEQ ID NO: 12 - DNA, 52 bp, Mentha x piperita
acgatactaggcggaatctcgacaacttattgaatccacacacccttgcagc

SEQ ID NO: 13 - DNA, 52 bp, Mentha x piperita
acgttatcaacgacagacacgtatatttcatccacgaacacacccttttggc

SEQ ID NO: 14 - DNA, 52 bp, Mentha x piperita
ataatgcgtggcagaaacatcaatctccattccgtgaacacacccttgtgtc

SEQ ID NO: 15 - DNA, 52 bp, Mentha x piperita
gcgagatctagggggatcttgactattccacgcgcgcacacacccttttgtg

SEQ ID NO: 16 - DNA, 52 bp, Mentha x piperita
ataagatgagccggagacatcaatactcaatccgtcaacacacccttgcgtc

SEQ ID NO: 17 - DNA, 52 bp, Mentha x piperita
atgatgcctaggagaaaaaccacaccttcatcagcccacacacccttgcgtg

SEQ ID NO: 18 - DNA, 52 bp, Mentha x piperita
acaataccaacgagagaaacgactacctcttgcacgcacaactttttgcatg